(19) United States
(12) Patent Application Publication
(10) Pub. No.: US 2012/0301409 A1
(43) Pub. Date: Nov. 29, 2012

(54) HOMEOPATHIC COMPOSITION AND METHOD FOR THE TREATMENT OF SKIN IRRITATIONS AND OTHER SKIN DISEASES

(76) Inventors: Ned L. Jensen, Alpine, UT (US); Peter E. Fuller, West Jordan, UT (US)

(21) Appl. No.: 13/113,226

(22) Filed: May 23, 2011

(51) Int. Cl. A61K 33/36; A61K 33/42; A61K 33/04; A61K 33/34; A61K 33/30; A61P 17/00; A61K 36/61; A61K 36/71; A61K 36/328; A61K 36/76; A61K 36/53; A61K 36/32; A61K 33/14; A61K 33/28

(52) U.S. Cl. 424/59; 424/680; 424/679; 424/601; 424/705; 424/630; 424/641; 424/620; 424/644; 424/725; 424/726; 424/742; 424/748; 424/769

(57) ABSTRACT

A homeopathic composition and method for treating a variety of skin irritations, wounds, and diseases is disclosed. The homeopathic composition may contain as active ingredients salicyclic acid, trona or one of its salts, and at least one homeopathic agent.

### BACKGROUND OF THE INVENTION

The present invention generally relates to compositions and methods for the treatment of skin irritations. More specifically, the present invention relates to compositions and methods for treating skin ailments by administering to the affected area a therapeutically effective amount of a homeopathic composition containing salicyclic acid, akaline earth carbonates and bicarbonates, and a homeopathic agent.

In recent years, there has been an increased effort to reduce or eliminate the risk of sunburn (erythema) produced by certain wavelengths in the ultraviolet (UV) region of the spectrum. Unfortunately, there are still circumstances wherein the skin becomes exposed to UV radiation. Such exposure may, in some cases, cause sunburn that needs to be treated.

The application of petroleum-based compositions to the sunburned area does not produce a sufficiently large heat transference effect to remove the heat from the sunburned area. Furthermore, the petroleum-based compositions tend to produce a residue that needs to be subsequently cleansed from the tender and sensitive area of sunburned skin, causing further discomfort to the patient.

As can be seen, there is a need for a composition for treating sunburn that provides immediate pain relief to the patient while also helping to promote healing of the skin.

### SUMMARY OF THE INVENTION

In one aspect of the present invention, a homeopathic composition for the treatment of skin irritations and other ailments includes salicylic acid, trona or one of trona salts, and at least one homeopathic ingredient.

In another aspect of the present invention, a method for treating a skin irritation or other ailments in a patient includes the step of topically or orally administering to the affected area a therapeutically effective amount of a homeopathic composition comprising a salicylic acid; trona or one of its salts; and at least one homeopathic agent.

In another aspect of the present invention, a method for maintaining the freshness of a product includes the steps of adding to an environment surrounding the product a homeopathic composition comprising salicylic acid; trona or one of its salts; and at least one homeopathic agent.

These and other features, aspects and advantages of the present invention will become better understood with reference to the following drawings, description and claims.

### BRIEF DESCRIPTION OF THE DRAWINGS

FIG. 1 illustrates a schematic diagram showing active ingredients used to prepare the composition according to an exemplary embodiment of the present invention; and

FIGS. 2-3 illustrate different bases on which the composition may be incorporated according to exemplary embodiments of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provides homeopathic compositions for treating a variety of skin irritations, wounds, and diseases by administering to the affected area a therapeutically effective amount of a homeopathic composition containing as active ingredients salicyclic acid, homeopathic agents, and akaline earth carbonates and bicarbonates.

As used herein, the term “skin irritation” may refer to any skin inflammation or itchiness causing discomfort to the patient. The skin irritation may be caused by burns, such as sunburn, heat burn, poisonous plants, allergens, insect bites, or puberty (acne); and wounds, such as lacerations.

As used herein, the term “therapeutically effective amount” is intended to refer to the amount of the composition necessary to bring relief to an affected tissue(s), organ(s), or organ system(s) of the patient.

As used herein, the term “topically administering” is intended to refer to the direct laying on, or spreading of, a composition on epidermal tissue at the affected site.

As used herein, the term “orally administering” is intended to refer to the direct administration by mouth to the affected individual.

As used herein, the term “homeopathic composition” is intended to refer to a composition for treating a disease based on the administration of minute doses of the agent that in massive amounts produces symptoms in healthy individuals similar to those of the disease itself. Thus, after a base preparation is made, either by an extract or maceration of an herbal compound or the dissolving of a selected compound in a solvent, a series of dilutions are prepared from the initial batch, called the “mother tincture”. Homeopathic compositions may be diluted according to either the decimal “X” or centesimal “C” scales. For a “3X HPUS” preparation, the mother tincture is diluted with nine parts of the desired diluent, in either liquid or powder form. The resultant mixture is then diluted a second time, in a ratio of one part mixture to nine parts solvent and the resulting mixture is diluted a third time in a ratio of one to nine. Therefore, the 3X drug is actually at 10⁻³ potency of the mother tincture. These scales are recognized by the Homeopathic Pharmacopeia of the United States (H.P.U.S.).

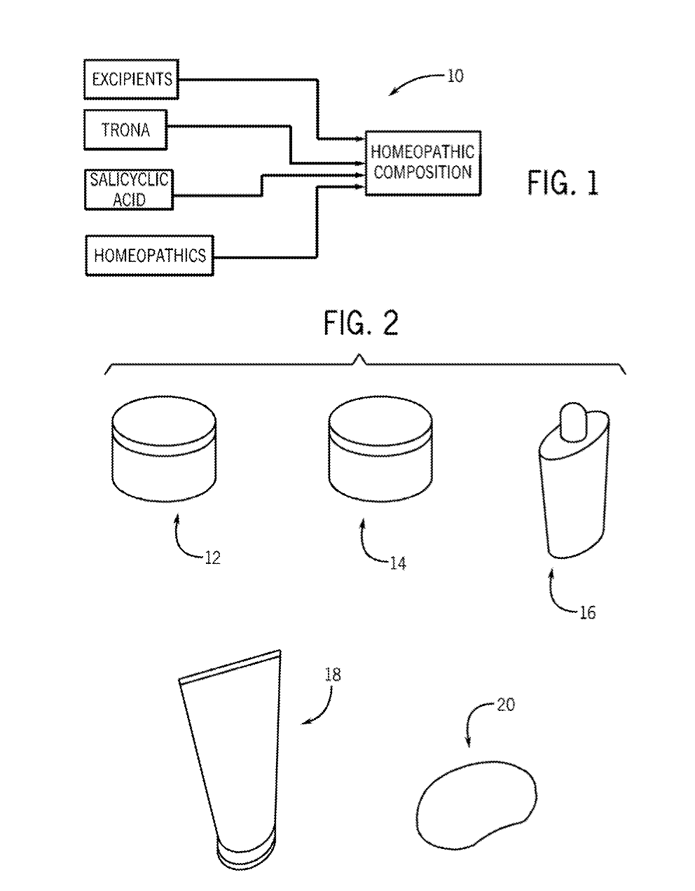

FIG. 1 illustrates active ingredients for a homeopathic composition 10 according to an exemplary embodiment of the present invention. The homeopathic composition 10 may include as active ingredients trisodium hydrogendicarbonate dihydrate (Trona), salicyclic acid, and at least one homeopathic agent.

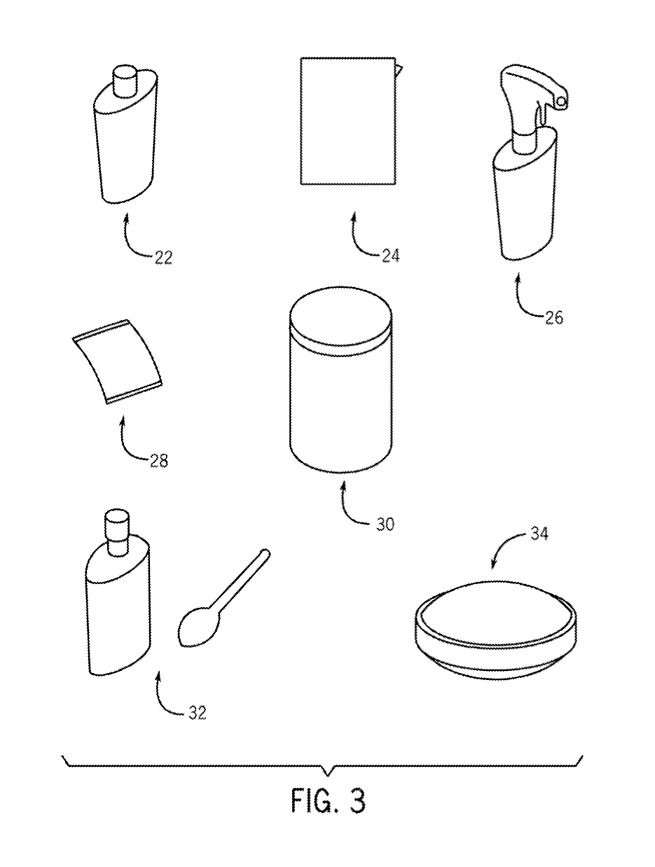

The homeopathic composition 10 may be incorporated into a base. FIGS. 2-3 illustrate that the base may be a cream 12, a paste 14, a liquid 16, a gel 18, a solid 20, a lotion 22, a powder 24, a spray 26, an ointment 28, an emulsion 30, oral solution 32, tablets 34, or capsules (not shown). The cream 12 may include, hand cream, shaving cream, antibacterial cream, acne cleanser, facial cleaning cream, or body cream. The paste 14 may include toothpaste, acne scrub, or facial cleansers. The liquid 16 may include liquid soaps, liquid detergents, liquid bubble bath soaps, food preservatives, face cleansers, food additives, liquid laundry detergent, or food preservation agents. The gel 18 may include toothpaste, acne topical gel, air refresher in gel form, food preservation agents, face cleansers, or soaps in gel form. The solid 20 may include soap, capsules, or pills. The lotion 22 may include body lotions, face cleansers, acne lotions, or shaving lotions. The powder 24 may include detergents, plant food additives, or food preservatives.

Trona is a natural mineral composed mainly of sodium carbonate, sodium bicarbonate, potassium carbonate and potassium bicarbonate with small amounts of magnesium and calcium salts and other trace minerals. The homeopathic composition 10 may use any commercially available form of trona including trona composite salts. The trona may be added to the homeopathic composition 10 for promoting the healing of the skin. The amount of trona in the homeopathic composition 10 may depend on the type of skin irritation to be treated. In some embodiments, the amount of trona present in the homeopathic composition 10 may be between 0.1 and 10% weight of the total composition 10. In some embodiments, the amount of trona present in homeopathic composition 10 may be 2.5% weight of the total homeopathic composition 10.

Salicylic acid may provide the homeopathic composition 10 with exfoliation and germicidal properties. Salicylic acid may include an organic acid that may chelate minerals such as iron, manganese and copper. This chelating ability may give the salicylic acid the ability to act as a cleaning agent when combined with Trona, various homeopathics, and sudsing agents. The amount of salicyclic acid on the homeopathic composition 10 may depend on the type of skin irritation to be treated. In some embodiments, the amount of salicyclic acid present in the homeopathic composition 10 may be between 0.1 and 10% weight of the total homeopathic composition 10.

Salicylic acid may irritate the skin of the patient, but when combined with Trona may produce a salt that may have a neutral pH that is gentle on the skin.

Homeopathic ingredients may be added to the homeopathic composition 10 to provide antibacterial, antifungal and antiviral activities. Homeopathic ingredients may include at least one of sodium chloride, potassium chloride, phosphorus, sulphur, silver, copper, zinc, arsenic, mercury salts, cloves, barberry, goldenseal, witch hazel, wintergreen, thyme, frankincense, myrrh, eucalyptus, phenol or white willow (the natural source of salicylic acid). In addition, the homeopathic composition 10 may include as a homeopathic ingredient any homeopathic ingredient as outlined in the Homeopathic Pharmacopoeia of the United States and the British, German and Indian Homeopathic Pharmacopoeias.

Carriers may be added to the composition 10. The carriers may be water, glycerin, ethanol, propylene glycol, isopropyl alcohol, vegetable oils such as soybean, avocado, sunflower, sesame seed oil, peanut oil, or other nut oils, canola, almond, jojoba oil, olive oil, safflower oil, grape seed oil, cranberry seed oil, sea buckthorn oil, any fatty acid ester such as isopropyl myristate, capryllic alcohol and esters.

In addition to salicyclic acid and akaline earth carbonates and bicarbonates, the composition of the invention may further comprise at least one of acidifying agents, alkalizing agents, aerosol propellants, antimicrobial agents, antioxidants, buffering agents, chelating agents, coloring additives, dispersing agents, emollients, emulsifying agents, humectants, fragrances, masking agents, preservatives, sugars, sunscreen agents, surfactants, suspending agents, or thickening agents.

Acidifying and alkalizing agents may be added to obtain the desired pH of the homeopathic composition 10. The acidifying agents may be at least one of acetic acid, citric acid, glacial acetic acid, malic acid, or proprionic acid. The alkalizing agents may be edetates, potassium carbonate, potassium hydroxide, sodium borate, sodium carbonate, or sodium hydroxide.

Aerosol propellants may be used when the homeopathic composition 10 may be administered as an aerosol under pressure. The aerosol propellants may be an inert gas including, but not limited to, nitrogen, carbon dioxide, helium, or argon.

Anti-microbial agents may be used when the skin area to be treated may be prone to microbial infection, bacteria, fungal, or protozoa. Anti-microbial agents may be benzyl alcohol, potassium sorbate, sorbic acid, benzoic acid, butyl paraben, ethyl paraben, methyl paraben, propyl pareben, or sodium benzoate.

Antioxidants may be used to protect ingredients of the homeopathic composition 10 from oxidizing agents that are included within or come in contact with the composition 10. The antioxidants may be at least one of water-soluble antioxidants such as all poly phenols, propyl gallate, ascorbic acid, carnosine, n-acetyl-camosine, and cysteine hydrochloridem and oil-soluble antioxidants such as retinoids, retinoic acid, all carotenes, ascorbyl palmitate, butytlated hydroxyanisole, butylated hydroxytoluene, octyl gallate, dodecyl gallate, phenyl-alpha-napthyl-amine, tocotrienols, and tocopherols such as alpha-tocopherol.

Buffering agents may be used to maintain an established pH for the composition 10. The buffering agents may be at least one of sodium citrate, calcium acetate, potassium metaphosphate, potassium phosphate monobasic, or tartaric acid.

Chelating agents may be used to maintain the ionic strength of the homeopathic composition 10 and/or bind to destructive compounds and metals that may be included within or come in contact with the homeopathic composition 10. The chelating agents may be at least one of dihydroxy ethyl glycine, amino acids, citric acid, tartaric acid, edetate dipotassium, edetate disodium, edetic acid, or ethylenediamine tetracetic acid (EDTA) and its salts (e.g., tetrasodium EDTA).

Coloring additives may be used to add color to the homeopathic composition 10. Coloring additives may be titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 10B, or Acid Red 51 or any of the FD&C approved colors designated for food and cosmetic use.

Dispersing and suspending agents may be quarternium-18 hectorite, polyhydroxy stearic acid, magnesium stearate, or other fatty acid salts, poligeenan, or silicon dioxide.

Emollient agents may soften and smooth the skin. The emollients may be at least one of hydrocarbon such as mineral oil, petrolatum, microcrystaline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholidds, or sterols.

Emulsifying agents may be used when the homeopathic composition 10 is an emulsion. The emulsifying agents used for preparing water-in-oil emulsions may be cyclomethicone (and) dimethicone copolyol, dimethicone copolyol, cetyl dimethicone copolyol, PEG-30 dipolyhydroxystearate, or PEG-40 sorbitan peroleate. The emulsifying agents used for preparing oil-in-water emulsions may be glyceryl stearate, PEG-100 stearate, methyl gluceth sesquisterate, fatty alcohols, or alkyl phenols condensed with ethylene oxide.

Humectants may promote the retention of moisture, e.g., moisturizers. The humectants may be at least one of sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl-1, 3-propanediol, and derivatives thereof, or hyaluronic acid.

Fragrances may be at least one of peppermint, rose oil, rose water, aloe vera, clove oil, menthol, camphor, or eucalyptus oil. Certain fragrances may require a solubilizer, e.g., PPG-5-ceteareth-20. To eliminate certain odors from compositions, masking agents may be used. An example of a masking agent may be ethylene brassylate.

Preservatives may be used to protect the homeopathic composition 10 from degradation. The preservatives may be phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, or phenol.

Sugars may be at least one of monosaccharides, disaccharides, and polysccharides such as glucose, xylose, fructose, reose, ribose, pentose, arabinose, allose, tallose, altrose, mannose, galactose, lactose, sucrose, erythrose, or glyceraldehyde.

Sunscreen agents may be added to the homeopathic composition 10 to block or reduce the amount of ultraviolet radiation impinging on the skin. The sunscreen agents may be organic compounds and/or inorganic materials. The organic compounds may be at least one of octyl methoxycinnamate, octyl salicylate, benzophenone-3 homosalate, octocrylate, avobenzone, or menthyl anthranilate. The inorganic materials may be at least one of zinc oxide, silica, iron oxide, titanium dioxide, or 2-ethyl-hexyl-p-methoxycinnamate.

Surfactants may be used to stabilize the homeopathic compositions 10. The surfactants may be used as wetting agents, antifoam agents, emulsifiers, dispersing agents, or penetrant agents. The surfactants may include at least one of methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, polysorbate 85, sodium lauryl sulfate, or sorbitan and its derivatives.

At least one humectant may be included when the homeopathic composition 10 is an aqueous composition. The humectant may reduce the tackiness produced by the aqueous composition after it has been applied and has dried on the skin. The humectants may be present in an amount such as to produce an aqueous composition that is capable of being substantially non-tacky after drying on the skin. The humectants may be at least one of glycerin, cellulose, or poly saccarides. The amount of humectants on the homeopathic composition 10 may be from about 0.1 to about 20% weight of the composition 10. In some embodiments, the amount of humectants on the homeopathic composition 10 may be 5% weight.

The ingredients may be mixed and/or blended together by using standard techniques well known in the art in order to manufacture the composition 10.

After mixing all of the ingredients, the homeopathic composition 10 may be packaged into the appropriate container so that the composition may be conveniently delivered and applied when needed. The container may be a tube, a bottle, a single-dose packet, box, sheet, or sprayer. In addition, the diluent composition 10 may be packed in single-dose packages to reduce the risk of contamination from one usage to the next.

### EXAMPLE

### USE OF AQUEOUS COMPOSITION TO TREAT SUNBURN

A 19-year old male and a 17-year old female, each suffering from classical erythema of the arms and face, were subjected to topical administration of an aqueous composition comprising of 2.5% salicylic acid, 5%, Trona at 2.5% glycerin, and the balance of water, and were monitored for skin surface temperature changes. The temperature was measured by using a cooper laser thermometer.

The initial skin surface temperatures prior to administration of the skin spray was:

on the arms for the male—96° F. and for the female—94° F.

on the face for the male—98° F. and for the female—96° F.

The skin surface temperatures about 90 seconds following administration of the homeopathic composition 10 was:

on the arms for the male—86° F. and for the female—86° F.

on the face for the male—85° F. and for the female—85° F.

The results show a drop of over 10° F. in a minute and a half when using the composition 10. In addition, it was noted that the pain on the area of the skin where the homeopathic composition 10 was applied was completely reduced in the same period and the skin was not sensitive to touch.

After a long search and research, the present inventors believe that the decrease in skin surface temperature may be associated with the production of carbon dioxide by the reaction of salicyclic acid with the akaline earth carbonates and bicarbonates in the presence of water. Carbon dioxide is a greenhouse gas, and is therefore effective for absorbing heat. Salicylic acid has anti-inflammatory properties which leads to a chemical change in the skin which may reduce the amounts of prostaglandins produced by cyclo-oxigenase in the skin.

Furthermore, the present inventors believed that the net result of combining salicyclic acid with the akaline earth carbonates and bicarbonates produces a composition with cooling properties.

### CREAM BASED FORMULA

A cream based formula may be made which also reduces inflammation and promotes healing and circulation. The cream based formula may contain appropriate amounts of deionized water, cetyl alcohol, glyceryl monostearate, isopropyl myristate, stearyl alcohol, avocado oil, sunflower oil, propylene glycol, glycerine, Vitamin E, lipoic acid, sodium benzoate, potassium sorbate, urea USP, polysorbate 20, xanthan gum, appropriate fragrances and MSM. To this the active ingredients of salicylic acid 5%, Trona 2.5%, and homeopathics Aloe Vera 3x, Arnica 4x, Calendula 4x, l Aesculus Hipp 4x, Berberis Vulgaris 4x , Zinc Sulfuricum 3x.

The patients that applied the cream based formulation of the present invention, to treat areas of the skin with burns and cuts, noted an immediate reduction of the pain, stopping of the bleeding, and reduction of the redness.

Some patients note an increasing sensation of feeling and circulation when the cream based formulation of the present invention was applied to their feet.

### ANTIBACTERIAL CREAM

Silver and copper were added at 4 times the strength to yield a cream with marked antibacterial activity.

### ACNE SCRUB

The homeopathic composition 10 may further include 10% cosmetic grade pumice, silver at 4x and copper at 4x to produce an acne scrub. Patients that used the acne scrub noticed an immediate clearing of acne and a definite reduction of redness and a marked improvement in skin appearance.

### ACNE CLEANSER

A liquid soap formulation was made from the following ingredients: liquid castile soap, imidurea, sodium benzoate, propylene glycol, Vitamin E, glycerine, polysorbate 20, and potassium sorbate. Then, a diluent composition 10 including salicylic acid 5%, Trona 2.5% following homeopathics Aloe 3x, Silver 4x, Cloves 3x, Lavender 3x, Menthol 3x, Rosemary 3x, and Thyme 4x was incorporated into the liquid soap formulation to obtain a cleanser.

The cleanser left a clean and very soft skin on the patients. The menthol and aloe increased circulation and left the skin cool and refreshed.

### ACNE TOPICAL GEL

The present invention manufactured a blue translucent gel with herbal fragrance. The gel included distilled water (DI water), sodium carboxymethylcellulose, glycine, sodium benzoate, imidurea, potassium sorbate, benzyl alcohol in a base of the following homeopathics: Salicylic Acid 2x, Sliver 4x, Zinc 3x, x, Calendula 3x, Aloe 3x, Aconite 12x, Apis Mel 12x, Cantharis 12x, Acidum Carbolicum 2x, Borax 3x, Natrium Carb (Trona Salt) 2x.

This gel showed astringent and cooling properties. The gel was well absorbed by the skin and did not leave any residue.

### BAR SOAP

The bar soap according to the present invention may be a white to off-white color vegetable soap that gives a high full lather and leaves the skin clean and fragrant. The soap was manufactured by combining 360 grams of coconut oil, 360 grams of olive oil, 600 grams of vegetable shortening, 480 grams of DI water, and 200 grams of refined trona salt (sodium carbonate). The oils were heated to 50° C. and set aside. The trona was placed in water and heat to 50° C. and stirred until clear. 15 grams of salicylic acid were added to the heated trona. The heated oils were combined with the trona and salicylic acid mixture and stirred until the soap began to thicken. 5 grams of Lavender and 5 grams of Lime oil were added and stirred in. The mixture was placed into soap molds and cured for 48 hours.

In some embodiments, the soap was grinded, added to powdered bubble bath and laundry soap mixture to make all natural soaps.

### BUBBLE BATH

A powdered soap was manufactured and combined with powdered trona or trona salts, salicylic acid at 1% and the following homeopathics: lavender 3x, orange oil 3x, and chamomile 3x, food colors, sudsing agents sodium laureth sulfate and cocamido-sulfosuccinate and other fragrances.

In some embodiments, the powdered soap may be substituted for the sudsing agents listed above to make an all natural bubble bath.

### LAUNDRY DETERGENT

A powdered soap composed of powdered Trona or Trona salts, salicylic acid at 1% and the following homeopathics: lavender 3x, lemon 3x, lime 3x. Food colors, magnesium peroxide, sudsing agents: sodium laureth sulfate and cocamido sulfosuccinate and other fragrances. Powdered soap may be substituted for the sudsing agents listed above to make an all natural laundry detergent.

### PLANT SPRAY

A liquid spray containing Trona salts at 1% and salicylic acid at 2% in a base of DI water, propylene glycol, benzyl alcohol, imidurea, sodium proprionate, and vitamin B1.

This spray makes flowers and vegetables stay fresh weeks longer.

### FLOWER VASE POWDERED ADDITIVE

A powder that may be added to the water that cut flowers are placed in. The formula should include trona salts, salicylic acid at 5%, and vitamin B1 at 0.1% with sodium proprionate and sodium benzoate as a preservative. Use would be ½ teaspoon per quart of water. This additive makes flowers stay fresh weeks longer.

### FOOD FRESHNESS EXTENDER

A powder comprising salicylic acid and trona from 1% salicylic acid to 90% salicylic acid that may be placed in the environment of freshly ground or cut meat including beef, pork, poultry and all types of seafood. This powder can be enclosed in breathable packaging such as slotted polyethylene plastic containers or sealed Tyvek film pouches. This powder may be added to water at the ratio of 1% to saturation and poured on absorbent pads and then sealed in polyethylene containers or sealed in Tyvek pouches.

This invention also entails the use of a two component system composed of an aqueous matrix sealed inside a Tyvek pouch that when punctured wets an absorbent pad. This then reacts and releases carbon dioxide and gaseous salicylic into the environment to enhance the freshness of the enclosed foods. Foods may be enclosed in single use packaging or in recirculating refrigeration display cases or even closed walk-in coolers.

### EXAMPLE OF USE OF INVENTION IN MAINTAINING FOOD FRESHNESS

A retail store in Salt Lake City, Utah allowed to test the homeopathic composition 10 by placing pouches behind their produce in their display cases and their walk-in coolers. One 70 gram packet was used for every lineal foot and placed two packets attached to the discharge vent of each cooler in the walk-in coolers. It was found that the produce losses due to spoilage went from $95 per day to $40 per day over a period of 60 days. Fruit losses went from $34 per day down to $12 per day and other produce went from $57 per day down to $28 per day. On a percentage basis the total losses went from 13% of gross sales down to 3% of gross sales losses during this 60 day period. This test was conducted between Oct. 1, 2010 and Dec. 1, 2010. The packets were changed out every thirty days because gas production drops after one month. The produce manager said that the crates of produce looked just as fresh in the cooler after 5 days as the new produce that was delivered that day.

### EXPECTORANT COUGH SYRUP

A cherry flavored with a soothing effect on sore throat and natural expectorants to break up mucus. The formula consists of alcohol/sugar syrup with cherry flavor to which the following homeopathics are added: Humulus 4x, Phosphorus 12x, Spongia 5x, Ammonium Bromide 3x, Ephedra 4x, Kali Carb(Trona Salt) 12x, Arsenicum 8x, Anise 4x, Menthol 3x, and Salicylic acid 2x, and Natrium Carb(Trona Salt) 2x.

### SORE THROAT LOZENGES

A chewable lozenge with an orange flavor. It consists of the following: fructose, dextrose, sterotex, magnesium stearate, orange flavor, orange food color, citric acid, Vitamin C 100 mg to which the following homeopathics are added: Zinc 5 mg, Silver 4x, Belladonna 12x, Rhus Tox 12x, Sticta 12x, Drosera 6x, Spongia 6x, Cinnaberris 12x, Salicylic Acid 2x, Natrium Carb(Trona Salt) 2x.

It should be understood, of course, that the foregoing relates to exemplary embodiments of the invention and that modifications may be made without departing from the spirit and scope of the invention as set forth in the following claims.

What is claimed is:

1. A homeopathic composition for the treatment of skin irritations and other ailments comprising:
salicylic acid;
trona or one of trona salts; and
at least one homeopathic ingredient,
wherein the homeopathic composition is adapted to be added to a base.

2. The homeopathic composition of claim 1, wherein the base is selected from creams, pastes, liquids, gels, solids, lotions, powders, sprays, ointments, emulsions, oral administrating solutions, tablets, or capsules.

3. The homeopathic composition of claim 1, wherein the base is selected from hand creams, shaving creams, antibacterial creams, acne cleansers, facial cleaning creams, body creams, toothpastes, acne scrubs, facial cleansers, liquid soaps, liquid detergents, liquid bubble bath soaps, food preservatives, face cleansers, food additives, liquid laundry detergent, food preservation agents, acne topical gels, air refreshers in gel form, food preservation agents, face cleansers, soaps in gel form, bar soaps, capsules, pills, acne lotions, shaving lotions, detergents, plants food additives, or food preservatives.

4. The homeopathic composition of claim 1, wherein the salicyclic acid is present in about 0.0001 to 10% weight or 1x to 6x HPUS dilution.

5. The homeopathic composition of claim 1, wherein the trona or the trona salt is present in an amount about 0.001% to 90% weight of the total composition.

6. The homeopathic composition of claim 1, further including at least one of acidifying agents, alkalizing agents, antimicrobial agents, antioxidants, buffering agents, chelating agents, color additives, dispersing agents, emollients, fragrances, masking agents, preservatives, sugars, sunscreen agents, surfactants, or suspending agents.

7. The homeopathic composition of claim 1, wherein the salicyclic acid present in the homeopathic composition in an amount between 0.1 to and 10% weight of the total homeopathic composition.

8. The homeopathic composition of claim 1, wherein the homeopathic agent includes at least one of sodium chloride, potassium chloride, phosphorus, sulphur, silver, copper, zinc, arsenic, mercury salts, cloves, barberry, goldenseal, witch hazel, wintergreen, thyme, frankincense, myrrh, eucalyptus, or white willow.

9. A method for treating a skin irritation or other ailments in a patient, the method comprising the step of:
topically or orally administering to an affected area a therapeutically effective amount of a homeopathic composition comprising salicylic acid; trona or one of its salts; and at least one homeopathic agent.

10. The method of claim 9, wherein the patient is a human or an animal.

11. A method for maintaining the freshness of a product, the method comprising the steps of adding to an environment surrounding the product a homeopathic composition comprising salicylic acid; trona or one of its salts; and
at least one homeopathic agent.

12. The method of claim 11, wherein the product is selected from candy, vegetables, flowers, meats, or baked goods.
